(19) United States
(12) Patent Application Publication
(10) Pub. No.: US 2026/0220759 A1
(43) Pub. Date: Jul. 30, 2026

(54) METHOD FOR EVALUATING FILTRATION FILTER

(71) Applicant: NSK LTD., Tokyo (JP)

(72) Inventors: Takenobu INABA, Kanagawa (JP); Yuya WATANABE, Kanagawa (JP)

(73) Assignee: NSK LTD., Tokyo (JP)

(21) Appl. No.: 19/151,917

(22) PCT Filed: Jan. 26, 2024
(86) PCT No.: PCT/JP2024/002425

§ 371 Date: Jul. 29, 2025

(30) Foreign Application Priority Data
Feb. 1, 2023 (JP) 2023-014037

(51) Int. Cl. G06T 7/00; A47J 37/12; B01D 35/143; F16N 39/06; G01N 21/25; G01N 33/03; G06T 7/90; H04N 23/88

(52) U.S. Cl. CPC G06T 7/0004; B01D 35/143; G01N 21/251; G01N 33/03; G06T 7/90; H04N 23/88; A47J 37/1223; F16N 39/06; G06T 2207/10024; G06T 2207/30128

(57) ABSTRACT

A method for evaluating a filtration filter used for recycling used fats and oils includes: an imaging step of capturing an image of fats and oils filtered by the filtration filter or an image of the fats and oils diluted with a solvent together with a color chart by an imaging device; an image information acquisition step of acquiring image information of the imaged fats and oils or image information of the fats and oils diluted with the solvent based on image information of the imaged color chart; a fats and oils deterioration determination step of determining a deterioration state of the fats and oils based on the image information; and a filtration filter evaluation step of evaluating filtration performance and deterioration inhibiting performance of the filtration filter based on a determination result of the fats and oils deterioration determination step.

### TECHNICAL FIELD

The present invention relates to a method for evaluating a filtration filter used for recycling fats and oils such as used lubricating oil and used edible oil.

### BACKGROUND ART

Edible oil is used for various foods, but edible oil used for cooking deep-fried foods such as tempura and fries oxidatively deteriorates when the edible oil is heated during cooking or left unattended, causing a taste, an odor, and an appearance of foods to deteriorate. The viscosity of oxidatively deteriorated oil increases, leading to poor drainage. Therefore, the edible oil that has oxidatively deteriorated to a predetermined level or higher is discarded. Hence, from the viewpoint of global environment conservation, it is desired to delay the oxidative deterioration in the edible oil as much as possible to reduce the number of times the edible oil is discarded. In addition, by reducing the number of times the edible oil is discarded, the number of times of cleaning a deep-frying utensil (fryer) used for deep-fried foods is reduced, and this adds the benefit that the amount of water used for cleaning can be reduced.

Various edible oil filtration filters are used to recycle used edible oil, and for example, Patent Literature 1 describes an edible oil filtration filter in which an adsorbent is uniformly dispersed by mixing fibers such as pulp and an adsorbent such as activated carbon powder with water by stirring, and dehydration performed by compression molding.

The filtration capability of the edible oil filtration filter decreases with the number of times of filtration, and therefore, it is necessary to determine the replacement time, and the number of times of use is generally specified in consideration of safety. However, deterioration states differ for each used edible oil, and the filtration capabilities of the edible oil filtration filter also differ accordingly. Therefore, the designated number of times of use is not necessarily suitable.

In addition, in various mechanical devices, a filtration filter is used to recycle lubricating oil used in a lubricating location such as a rotation location, but there is a similar problem.

As a method for evaluating the filtration capability of a filtration filter, for example, in Patent Literature 2, a dispersion of metal particles is filtered through a filter, and a filtrate is sampled. The metal particles are dissolved in a chemical solution to form a solution, and the solution is analyzed by ICP-MS. The amount of the metal particles in the sampled filtrate is detected, and a filtration membrane is evaluated based on the detected amount.

### CITATION LIST

### Patent Literature

Patent Literature 1: JP4593127B
Patent Literature 2: JP6006541B

### SUMMARY OF INVENTION

### Technical Problem

However, in the evaluation method described in Patent Literature 2, a dispersion of metal particles is required, and only the collection performance of foreign substances in the filtrate can be evaluated. It is necessary to use ICP-MS during the evaluation, the device is large-scale. In addition, it is necessary to subject the filtrate to the ICP-MS, and the filtration performance cannot be immediately evaluated.

Therefore, an object of the present invention is to provide a method for evaluating a filtration filter, which is capable of easily evaluating filtration performance and deterioration inhibiting performance immediately on site in a small sampling amount without using a large-scale detection device.

### Solution to Problem

The above object of the present invention is achieved by the following configuration [1] related to a method for evaluating a filtration filter.
[1] A method for evaluating a filtration filter, which is a method for evaluating a filtration filter used for recycling used fats and oils, the method including:
an imaging step of capturing an image of fats and oils filtered by the filtration filter or an image of the fats and oils diluted with a solvent, together with a color chart, by an imaging device;
an image information acquisition step of acquiring image information of the imaged fats and oils or image information of the fats and oils diluted with the solvent based on image information of the imaged color chart;
a fats and oils deterioration determination step of determining a deterioration state of the fats and oils based on the image information; and
a filtration filter evaluation step of evaluating filtration performance and deterioration inhibiting performance of the filtration filter based on a determination result of the fats and oils deterioration determination step.

A preferred embodiment according to the present invention related to the method for evaluating a filtration filter relates to the following [2] to [6].
[2] The method for evaluating a filtration filter according to claim [1],
in which the image information in the image information acquisition step is an RGB value of the fats and oils after filtration or an RGB value of the fats and oils diluted with the solvent, and
in the fats and oils deterioration determination step, lightness (ΔE) defined by the following formula (1) and a maximum color difference are calculated from the RGB value to determine a correlation between the lightness (ΔE) and the maximum color difference which are defined as follows:

$$Lightness ( Δ E ) = {( {R}^{2} + {G}^{2} + {B}^{2} )}^{0.5} ; \qquad ( 1 )$$

Maximum color difference: difference between maximum value and minimum value in RGB values.
[3] The method for evaluating a filtration filter according to [2], further including:
an image information correction step of correcting a white balance in the image information of the imaged fats and oils based on a white balance in the image information of the color chart, in a subsequent stage of the image information acquisition step,
in which in the fats and oils deterioration determination step, the deterioration state of the fats and oils is determined based on the corrected image information.
[4] The method for evaluating a filtration filter according to [3], in which the imaging device has a white balance correction function.
[5] The method for evaluating a filtration filter according to [4], in which the imaging device is a digital camera or a camera-equipped mobile terminal.
[6] The method for evaluating a filtration filter according to any one of [1] to [5], in which the fats and oils is lubricating oil or edible oil.

### Advantageous Effects of Invention

According to the method for evaluating a filtration filter of the present invention, it is possible to easily evaluate filtration performance and deterioration inhibiting performance immediately on site in a small sampling amount without using a large-scale detection device.

### BRIEF DESCRIPTION OF DRAWINGS

FIG. 1 is a top view illustrating an example of a color chart.

FIG. 2 is a schematic view illustrating a device used in Test 1 and Test 2.

FIG. 3 is a graph illustrating results of a commercially available filter in Test 1.

FIG. 4 is a graph illustrating results obtained by a deterioration inhibiting filter in Test 1.

FIG. 5 is a graph illustrating results obtained by a commercially available filter in Test 2.

FIG. 6 is a graph illustrating results obtained by a deterioration inhibiting filter in Test 2.

### DESCRIPTION OF EMBODIMENTS

Hereinafter, an embodiment of the present invention will be described in detail with reference to the drawings. It should be noted that the present invention is not limited to the embodiments described below, and can be freely modified and implemented without departing from the gist of the present invention.

### (Imaging Step)

In a method for evaluating a filtration filter of the present invention (hereinafter referred to as “evaluation method”), first, a device or equipment using fats and oils such as lubricating oil and edible oil, for example, a rolling bearing, a ball screw device, or a fryer is stopped, the fats and oils is sampled and taken into a container, and is imaged together with a color chart. Various imaging devices can be used for image capturing, and the type thereof is not particularly limited. For example, a digital camera or a camera-equipped mobile terminal such as a smartphone and a tablet can be used. The type of a light source during image capturing is not particularly limited, and detection can be performed at various places.

### (Image Information Acquisition Step)

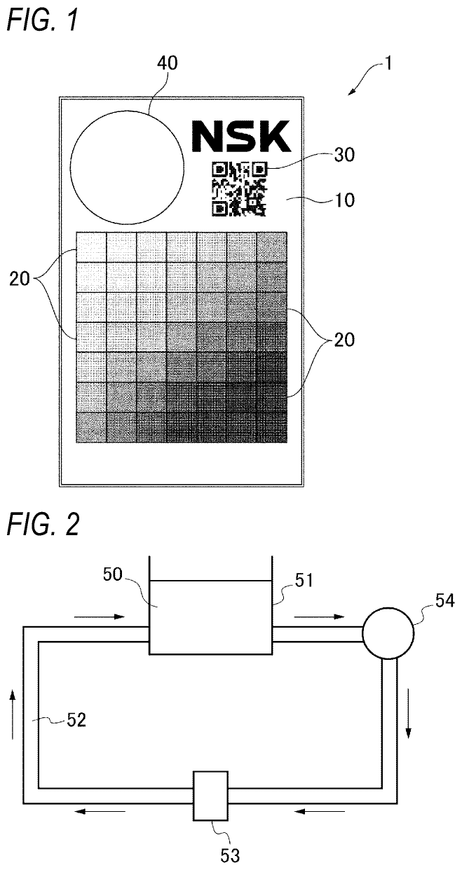

In this step, a color chart is used. As illustrated in FIG. 1, a color chart 1 is a list in which a plurality of color samples 20 having different hues and shades from white (upper left in the drawing) to black (lower right in the drawing) are arranged on a surface of a base sheet 10. The image information of the color chart 1 is printed on the base sheet 10 as an identification code 30. As the identification code 30, a bar code, a QR code (registered trademark) illustrated in the drawing, or the like is used.

Then, the sampled fats and oils is charged into a container (not illustrated) such as a petri dish or a transparent bin, placed on a sample placement portion 40 indicated by a circle in the drawing, and imaged together with the color chart 1 using various imaging devices. Here, a sampling amount of the fats and oils is about 10 mg, which is a trace quantity. The fats and oils may be used alone or may be diluted with a solvent. The solvent is not particularly limited as long as it is easily mixed with the fats and oils and has a colorless property and transparency to such an extent that there is no problem in capturing an image of the color of the fats and oils. Specifically, organic solvents, kerosene, and gasoline are preferable. The fats and oils discolored in a dark color, particularly in a black color can be more finely separated by diluting the fats and oils with a solvent.

### (Image Information Correction Step)

The image information of the imaged fats and oils is compared with the image information of the color chart 1. At this time, it is preferable to correct the white balance in the image information of the imaged fats and oils. When the white balance is corrected, it is possible to make the image information of the imaged fats and oils more appropriately correspond to the image information of the color chart 1 regardless of the environment of the imaging location, that is, the detection location, which may be affected by brightness or the like.

When the imaging device to be used has a white balance correction function, the white balance correction can be performed by the white balance correction function. Alternatively, the image information obtained by the imaging device may be transmitted to an external processing device such as a server, and correction may be performed by a white balance correction function of the processing device. The comparison between the image information of the lubricant and the image information of the color chart 1, which will be described below, may be performed in the imaging device or may be performed in an external processing device.

### (Fats and Oils Deterioration Determination Step)

Next, a hue of an image of the fats and oils with corrected white balance (hereinafter referred to as “corrected image”) is determined. The hue is expressed by three colors, red (R), green (G), and blue (B), and lightness (ΔE) is determined from the following formula (1) based on the RGB values of the corrected image.

$$Lightness ( Δ E ) = {( {R}^{2} + {G}^{2} + {B}^{2} )}^{0.5} \qquad ( 1 )$$

A difference between the maximum value and the minimum value of the RGB values is the maximum color difference of the corrected image of the lubricant.

As shown in test examples to be described below, when the lightness (ΔE) and the maximum color difference are determined for each predetermined filtration time or for each number of times of filtration, and the lightness (ΔE) and the maximum color difference are plotted in an X axis and a Y axis, respectively, and graphed, a semicircular arc-shaped correlation is observed. Based on this graph, the degree of deterioration (deterioration state) of the collected fats and oils is determined.

In the case where the fats and oils is diluted, the correction based on a dilution ratio can be performed using a correction table or the like created in advance through the correction function implemented in the imaging device or the server.

### (Filtration Filter Evaluation Step)

Then, the filtration performance and the deterioration inhibiting effect of the filtration filter are evaluated based on the deterioration state of the fats and oils.

### EXAMPLES

Hereinafter, the present invention will be further clarified with reference to Examples and Comparative Examples.

### <Test 1: Evaluation of Continuous Filtration>

A commercially available filter was prepared as a comparative example, and a deterioration inhibiting filter was prepared as an example. The deterioration inhibiting filter is provided with an effect of inhibiting deterioration in oil by causing an antioxidant to adhere thereto.

Each filter was attached to the device shown in FIG. 2, and the deterioration state of rapeseed oil was examined. The illustrated device includes an oil tank 51 in which rapeseed oil 50 is stored, an annular pipe 52 through which the rapeseed oil 50 passes, a pump 54 that feeds the rapeseed oil 50, and a processing unit 53 in which a filtration filter is loaded. The rapeseed oil 50 in the oil tank 51 is fed through the pipe 52 by the pump 54, filtered in the processing unit 53, and then returned to the oil tank 51.

Then, the rapeseed oil 50 was continuously filtered while being heated to 180° C., and the filtered rapeseed oil 50 was collected after 24 hours, 48 hours, and 96 hours. The sampling amount was 1 g each time.

The sampled rapeseed oil was charged in a petri dish and placed on the sample placement portion 40 of the color chart 1 shown in FIG. 1, the sampled rapeseed oil and the color chart were imaged together by a digital camera with a white balance correction function, and RGB values were determined from the obtained corrected image. Then, the lightness (ΔE) and the maximum color difference were calculated from the obtained RGB values based on the formula (1), and a graph in which the lateral axis represents the lightness (ΔE) and the vertical axis represents the maximum color difference was created.

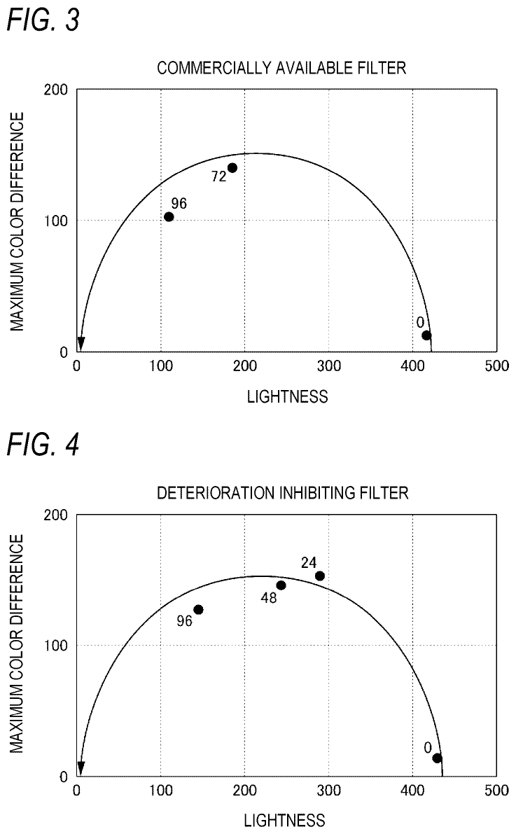

FIG. 3 is a graph showing the lightness (ΔE) and the maximum color difference for each filtration time in the case of filtration with a commercially available filter, and FIG. 4 is a graph showing the lightness (ΔE) and the maximum color difference for each filtration time in the case of filtration with a deterioration inhibiting filter. As shown in the drawings, in both filters, the rapeseed oil has almost no color and high lightness when it is unused oil. However, as the filtration time elapsed, a continuous change was observed in which the lightness decreased because the filtered rapeseed oil deteriorated and became brown, and the maximum color difference increased because the hue decreased. In addition, when the commercially available filter and the deterioration inhibiting filter are compared after 96 hours of filtration, it can be seen that the rapeseed oil filtered by the commercially available filter has both a lower lightness (ΔE) and a lower maximum color difference than the rapeseed oil filtered by the deterioration inhibiting filter, and the deterioration has progressed.

### <Test 2: Evaluation of Number of Times of Filtration>

The deteriorating rapeseed oil was filtered separately using a commercially available filter and a deterioration inhibiting filter. At this time, only oil was dropped from each filter, and the device shown in FIG. 2 was not used. Then, filtration was performed once or three times, and a change in hue of the rapeseed oil after filtration was observed. The sampling amount was 1 g each time, which is the same as that of Test 1.

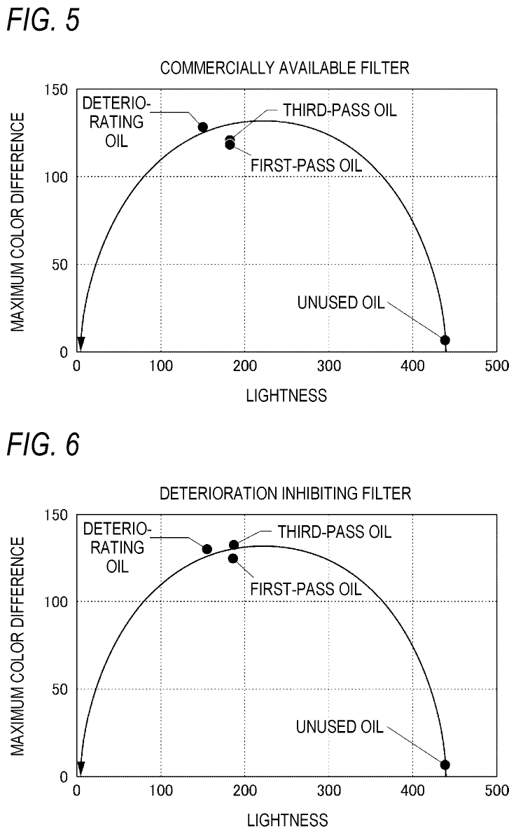

The hue measurement was performed in the same manner as in Test 1, the lightness (ΔE) and the maximum color difference were calculated from the determined RGB values, and a graph in which the lateral axis represents the lightness (ΔE) and the vertical axis represents the maximum color difference was created. FIG. 5 is a graph showing the lightness (ΔE) and the maximum color difference for each number of times of filtration in the case of filtration with a commercially available filter, and FIG. 6 is a graph showing the lightness (ΔE) and the maximum color difference for each number of times of filtration in the case of filtration with a deterioration inhibiting filter. The “deteriorating oil” in FIGS. 5 and 6 indicates a measurement value for rapeseed oil that has not been filtered even once.

As shown in the drawings, in both filters, the rapeseed oil has almost no color and high lightness when it is unused oil. However, as the number of times of filtration increases, the lightness decreases because the filtered rapeseed oil deteriorates and becomes brown. In addition, when the commercially available filter and the deterioration inhibiting filter are compared after filtration is performed three times, it can be seen that the rapeseed oil filtered by the deterioration inhibiting filter has a larger maximum color difference, and has inhibited deterioration.

It is difficult to visually confirm the change in hue as shown in Test 1 and Test 2, and it is difficult to determine the deterioration state of the filtration filter. However, accurate evaluation can be performed by the evaluation method according to the present invention.

Although various embodiments have been described above, it is needless to say that the present invention is not limited to these examples. It is apparent to those skilled in the art that various changes or modifications can be conceived within the scope described in the claims, and it is understood that the changes or modifications naturally fall within the technical scope of the present invention. In addition, the components described in the above embodiments may be combined in any manner without departing from the spirit of the invention.

The present application is based on a Japanese patent application (No. 2023-014037) filed on Feb. 1, 2023, contents of which are incorporated herein by reference.

### REFERENCE SIGNS LIST

1: color chart
10: base sheet
20: color sample
30: identification code
40: sample placement portion
50: rapeseed oil
51: oil tank
52: pipe
53: processing unit
54: pump

1. A method for evaluating a filtration filter, which is a method for evaluating a filtration filter used for recycling used fats and oils, the method comprising:
an imaging step of capturing an image of fats and oils filtered by the filtration filter or an image of the fats and oils diluted with a solvent, together with a color chart, by an imaging device;
an image information acquisition step of acquiring image information of the imaged fats and oils or image information of the fats and oils diluted with the solvent based on image information of the imaged color chart;
a fats and oils deterioration determination step of determining a deterioration state of the fats and oils based on the image information; and
a filtration filter evaluation step of evaluating filtration performance and deterioration inhibiting performance of the filtration filter based on a determination result of the fats and oils deterioration determination step.

2. The method for evaluating a filtration filter according to claim 1,
wherein the image information in the image information acquisition step is an RGB value of the fats and oils after filtration or an RGB value of the fats and oils diluted with the solvent, and
in the fats and oils deterioration determination step, lightness (ΔE) defined by the following formula (1) and a maximum color difference are calculated from the RGB value to determine a correlation between the lightness (ΔE) and the maximum color difference which are defined as follows:
$$Lightness ( Δ E ) = {( {R}^{2} + {G}^{2} + {B}^{2} )}^{0.5} ; \qquad ( 1 )$$
and
Maximum color difference: difference between maximum value and minimum value in RGB values.

3. The method for evaluating a filtration filter according to claim 2, further comprising:
an image information correction step of correcting a white balance in the image information of the imaged fats and oils based on a white balance in the image information of the color chart, in a subsequent stage of the image information acquisition step,
wherein in the fats and oils deterioration determination step, the deterioration state of the fats and oils is determined based on the corrected image information.

4. The method for evaluating a filtration filter according to claim 3, wherein the imaging device has a white balance correction function.

5. The method for evaluating a filtration filter according to claim 4, wherein the imaging device is a digital camera or a camera-equipped mobile terminal.

6. The method for evaluating a filtration filter according to claim 1, wherein the fats and oils is lubricating oil or edible oil.
